(19) United States
(12) Patent Application Publication
(10) Pub. No.: US 2005/0002922 A1
(43) Pub. Date: Jan. 6, 2005

(54) Use of secretin and secretin analogs to treat affective disorders

(76) Inventors: Richard Boismenu, Ashland, MA (US); James R. Rusche, Framingham, MA (US); Martin Goulet, Waltham, MA (US)

Correspondence Address: FISH & RICHARDSON PC, 225 FRANKLIN ST, BOSTON, MA, 02110, US

(21) Appl. No.: 10/800,344

(22) Filed: Mar. 12, 2004

Related U.S. Application Data
(60) Provisional application No. 60/454,421, filed on Mar. 12, 2003.

(51) Int. Cl. A61K 38/54

(52) U.S. Cl. 424/94.2

(57) ABSTRACT

The invention is based, in part, on the discovery that secretin and secretin analogs can be used to treat affective disorders that include symptoms of anxiety. Secretin and secretin analogs can be administered to patients to treat affective disorders, such as mood, psychotic, mental, substance-abuse withdrawal, eating, and sexual disorders, as well as other disorders with attendant sensory processing dysfunction, including symptoms of anxiety and/or fear. In addition, secretin and secretin analogs can be administered to improve cognition, including learning and memory, as well as to lessen the effects of conditioned startle. Secretin has the added benefit of causing no negative side effects upon administration and being easy to administer.

### CROSS-REFERENCE TO RELATED APPLICATION

This application claims the benefit of U.S. Provisional Patent Application Ser. No. 60/454,42 1, filed on Mar. 12, 2003, the contents of which is incorporated herein by reference in its entirety.

### FIELD OF THE INVENTION

This invention relates to the use of secretin and secretin analogs to treat various affective disorders.

### BACKGROUND OF THE INVENTION

Neuropsychiatric, neurological, and behavioral disorders, such as those associated with anxiety, have been difficult to understand and to treat effectively. Such disorders are serious medical illnesses that affect many millions of adults in the United States and abroad. These disorders affect people's emotions, feelings, sensibilities, and mental states, and can include symptoms of overwhelming anxiety and fear.

Most of the discovery effort for new neurologically active drugs to treat these disorders has been based on the study of agonist/antagonist drug interaction with one or more of the numerous neurotransmitter receptors in the brain, as well as their respective neurotransmitter receptor ligands (e.g., GABA, glutamate, and serotonin). In addition, researchers are finding new uses for known drugs. For example, a number of medications originally approved for the treatment of depression have been found to be effective for anxiety disorders. These medications include, but are not limited to, those belonging to the drug classes known as anticonvulsants, azaspirones, benzodiazepines, beta blockers, monoamine oxidase inhibitors (MAOIs), selective serotonin reuptake inhibitors (SSRIs), and tricyclic antidepressants (TCAs). However, these medications are often accompanied by unwanted side-effects.

### SUMMARY OF THE INVENTION

The invention involves the use of secretin and secretin analogs to treat emotional, behavioral, neurological, and mental disorders, collectively referred to herein as “affective disorders,” all of which may include anxiety as a symptom, as well as the use of secretin and its analogs as cognitive enhancers, for example, to enhance learning and memory.

Affective disorders include, for example, anxiety disorders (e.g., generalized anxiety disorder (GAD), social anxiety disorder (SAD; alternatively known as social phobia), panic disorder (with or without agoraphobia), posttraumatic stress disorder (PTSD), obsessive-compulsive disorder (OCD), separation anxiety disorder), mood disorders (e.g., depressive disorder, bipolar disorder) psychotic disorders (e.g., schizophrenia, schizoaffective disorder, delusional disorder), substance-related disorders (e.g., substance abuse, substance-induced disorder, substance withdrawal), cognitive disorders (e.g., dementia, delirium, Alzheimer's type dementia), affective disorder associated with neurological medical disorders (e.g., a seizure disorder, epilepsy), and affective disorders of childhood (e.g., attention disorder, attention deficit hyperactivity disorder, learning disorder, separation anxiety).

These affective disorders may all include symptoms of anxiety and decreased cognitive function (for example, decreased learning and memory). The invention is based, at least in part, on the discovery that secretin, a naturally occurring peptide originally implicated in gastrointestinal function, is a potent anxiolytic (i.e., anxiety reducing compound), can modify behavior, and enhance cognitive performance. Secretin and secretin analogs can thus be used to treat (e.g., reduce symptoms of) affective disorders as described herein.

In addition, secretin and secretin analogs can be used more generally to reduce anxiety and fear-potentiated startle in both humans and animals. Secretin and secretin analogs can also be used to enhance cognition, including learning and memory.

An important advantage of secretin and secretin analogs is their efficacy without adverse side-effects in a patient. In contrast, conventional antidepressants and antianxiety drugs typically lead to adverse side-effects that can include sedation, cognitive impairment, appetite stimulation, tardive dyskinesia (irreversible, involuntary movement disorder), extrapyramidal symptoms, and akathesia symptoms. Side effects are one of the major reasons for medical noncompliance in the outpatient treatment of affective disorders, such as anxiety disorders. Because they lack significant side effects, secretin and secretin analogs represent an improvement over those drugs that cause side effects.

Secretin and secretin analogs can be used alone or in conjunction with other medications, including other anxiolytic drugs.

The invention features methods for treating an affective disorder (e.g., generalized anxiety disorder (GAD), social anxiety disorder (SAD; alternatively known as social phobia), panic disorder (with or without agoraphobia), posttraumatic stress disorder (PTSD), obsessive-compulsive disorder (OCD), separation anxiety disorder, a mood disorder (e.g., depressive disorder or bipolar disorder), a psychotic disorder (e.g., schizophrenia, schizoaffective disorder, or delusional disorder), a substance-related disorder (e.g., substance abuse, substance-induced disorder, or substance withdrawal(e.g., where the substance is alcohol, an amphetamine, cocaine, nicotine, or an opioid)), a cognitive disorder (e.g., dementia, delirium, or Alzheimer's type dementia), an affective disorder associated with neurological medical disorders (e.g., a seizure disorder or epilepsy), or an affective disorder of childhood (e.g., attention disorder, attention deficit hyperactivity disorder, learning disorder, or separation anxiety) in a patient (e.g., human, dog, cat, horse, donkey, cow, sheep, goat, pig, rat, or mouse), in which the method includes administering (e.g., subcutaneously, orally, nasally, parenterally, or intravenously; e.g., multiple times, either at regular intervals or intermittently) to the patient a dosage (e.g., 0.04 μg/kg, 0.5 μg/kg, 1 μg/kg, 3 μg/kg, 10 μg/kg, 30 μg/kg, 100 μg/kg, or 400 μg/kg) of secretin (e.g., human, bovine, equine, porcine, or canine secretin) or a secretin analog effective to reduce one or more symptoms (e.g., anxiety, fear, impaired learning, impaired memory, apathy, delusions, anxiety, and autonomic changes, avoidance, increased arousal, depression, elevated mood, irritable mood, hallucinations, disorganized speech, and grossly disorganized behavior) of the affective disorder (e.g., in which the patient suffers no adverse side-effects).

The invention also features methods for reducing anxiety in a patient (e.g., a patient with generalized anxiety disorder (GAD), social anxiety disorder (SAD; alternatively known as social phobia), panic disorder (with or without agoraphobia), posttraumatic stress disorder (PTSD), obsessive-compulsive disorder (OCD), separation anxiety disorder, a mood disorder, a psychotic disorder, a substance-related disorder, a cognitive disorder, an affective disorder associated with neurological medical disorders, or an affective disorder of childhood.), in which the method includes administering (e.g., subcutaneously, orally, nasally, parenterally, or intravenously; e.g., multiple times, either at regular intervals or intermittently) to the patient (e.g., a mammal, such as a human, dog, cat, horse, donkey, cow, sheep, goat, pig, rat, or mouse, including a mammal kept in captivity) a dosage (e.g., about 0.04 μg/kg, 0.5 μg/kg, 1 μg/kg, 3 μg/kg, 10 μg/kg, 30 μg/kg, 100 μg/kg, or 400 μg/kg) of secretin (e.g., human, bovine, equine, porcine, canine, or another mammalian homolog) or a secretin analog (e.g., a peptide) effective to reduce anxiety (e.g., without causing adverse side effects).

The invention encompasses methods for treating a patient having a disorder (e.g., generalized anxiety disorder (GAD), social anxiety disorder (SAD; alternatively known as social phobia), panic disorder (with or without agoraphobia), posttraumatic stress disorder (PTSD), obsessive-compulsive disorder (OCD), separation anxiety disorder, a mood disorder (e.g., depressive disorder or bipolar disorder), a psychotic disorder (e.g., schizophrenia, schizoaffective disorder, or delusional disorder), a substance-related disorder (e.g., substance abuse, substance-induced disorder, or substance withdrawal(e.g., where the substance is alcohol, an amphetamine, cocaine, nicotine, or an opioid)), a cognitive disorder (e.g., dementia, delirium, or Alzheimer's type dementia), an affective disorder associated with neurological medical disorders (e.g., a seizure disorder or epilepsy), or an affective disorder of childhood (e.g., attention disorder, attention deficit hyperactivity disorder, learning disorder, or separation anxiety)) with one or more attendant symptoms of anxiety, impaired learning, or impaired memory in a patient (e.g., human, dog, cat, horse, donkey, cow, sheep, goat, pig, rat, or mouse), in which the method includes administering (e.g., subcutaneously, orally, nasally, parenterally, or intravenously; e.g., multiple times, either at regular intervals or intermittently) to the patient a dosage (e.g., 0.04 μg/kg, 0.5 μg/kg, 1 μg/kg, 3 μg/kg, 10 μg/kg, 30 μg/kg, 100 μg/kg, or 400 μg/kg) of secretin (e.g., human, bovine, equine, porcine, or canine 30 secretin) or a secretin analog effective to reduce one or more attendant symptoms of the disorder (e.g., in which the patient suffers no adverse side-effects).

The invention also features methods for enhancing cognition in a patient (e.g., a patient with generalized anxiety disorder (GAD), social anxiety disorder (SAD; alternatively known as social phobia), panic disorder (with or without agoraphobia), posttraumatic stress disorder (PTSD), obsessive-compulsive disorder (OCD), separation anxiety disorder, a mood disorder, a psychotic disorder, a substance-related disorder, a cognitive disorder, an affective disorder associated with neurological medical disorders, or an affective disorder of childhood; e.g., in which the patient suffers from impaired learning or memory), in which the method includes administering (e.g., subcutaneously, orally, nasally, parenterally, or intravenously; e.g., multiple times, either at regular intervals or intermittently) to the patient (e.g., a mammal, such as a human, dog, cat, horse, donkey, cow, sheep, goat, pig, rat, or mouse, including a mammal kept in captivity) a dosage (e.g., about 0.04 μg/kg, 0.5 μg/kg, 1 μg/kg, 3 μg/kg, 10 μg/kg, 30 μg/kg, 100 μg/kg, or 400 μg/kg) of secretin (e.g., human, bovine, equine, porcine, canine, or another mammalian homolog) or a secretin analog (e.g., a peptide) effective to enhance learning (e.g., in which enhancement or learning is measured by improvement on a Wisconsin Card Sort Test) or memory or both in the patient (e.g., without causing adverse side effects).

Another feature of the invention is a method for reducing fear-potentiated startle in a patient, in which the method includes administering to the patient (e.g., a mammal, such as human, dog, cat, horse, cow, sheep, goat, or pig) suffering from fear-potentiated startle a dosage of secretin or a secretin analog effective to reduce startle.

Also encompassed by the invention are compositions for use as medicaments in treating affective disorders, reducing anxiety, treating a disorder with one or more attendance symptoms of anxiety, impaired learning, and impaired memory, and enhancing cognition in a patient according any of the methods (and any of the details thereof) outlined herein.

The invention additionally includes uses of compositions for the manufacture of medicaments for treating affective disorders, reducing anxiety, treating a disorder with one or more attendance symptoms of anxiety, impaired learning, and impaired memory, and enhancing cognition in a patient according any of the methods (and any of the details thereof) outlined herein.

In addition, the invention includes kits that include a composition including secretin or a secretin analog and instructions for its use in treating affective disorders, reducing anxiety, treating a disorder with one or more attendance symptoms of anxiety, impaired learning, and impaired memory, and enhancing cognition in a patient according any of the methods outlined herein.

As used herein, the term “anxiolytic” means a substance capable of reducing anxiety in a subject (whether human or animal).

As used herein, the term “enhanced cognition” means an improvement in a mental function, such as learning or memory.

As used herein, the term “patient” or “subject” refers to an individual to whom, or animal, e.g., a mammal, such as a dog, cat, horse, cow, pig, goat, rat, or mouse, to which secretin or a secretin analog can be administered.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIGS. 1-3 are graphic representations of data gathered in the conduct of testing secretin in various animal models well accepted in the field of anxiety studies and spatial memory and learning.

FIG. 1 is a bar graph that depicts the effect on baseline startle amplitude of secretin administered to rats.

FIG. 2 is a bar graph that depicts the effect on expression of fear-potentiated startle of secretin administered to rats.

FIG. 3 is a table that depicts the effect on spatial memory and learning, measured in a water maze test, of secretin administered to rats.

FIG. 4 is an evaluation schedule for a clinical study of the treatment of Obsessive Compulsive Disorder with secretin. PK=pharmacokinetics. ConMeds=concommitant medications (that is, other drugs being taken by patients). AE=adverse events.

### DETAILED DESCRIPTION

The invention includes the use of secretin and secretin analogs to treat affective disorders, e.g., disorders with symptoms of anxiety and clinical consequences of impaired cognition. The invention is based, at least in part, on the discovery that secretin is useful as an anxiolytic. As a result, secretin and secretin analogs can be used to treat a variety of affective disorders involving anxiety or fear. Secretin and secretin analogs can also be administered to reduce fear-potentiated startle.

The invention also includes the use of secretin and secretin analogs to enhance cognitive performance. Cognition impairment is exemplified by learning and memory problems in Alzheimer's disease and social cognition (flat affect, ahedonia) in schizophrenia. Many drugs that are anxiolytic drugs do not show improvements in cognition. Secretin improves cognition in a functional learning and memory test. As an effective cognitive enhancer, secretin and secretin analogs can be used to improve cognition, and, thus, patient quality of life in a variety of disorders in which an anxious state may be contributing to the main symptoms of the disease. This includes anxiety disorders, mood disorders, psychotic disorders, substance-induced disorders, affective disorders of childhood, as well as disorders principally categorized as cognitive disorders. For example, secretin and secretin analogs can be administered to a patient with schizophrenia who has an inability to manage daily functions due to lack of memory, and secretin can induce improvement in cognition which results in clear clinical improvement.

Characteristics of Affective Disorders

The invention includes the use of secretin and secretin analogs to treat affective disorders to reduce, inhibit, or prevent their attendant symptoms such as anxiety and fear, resulting in comorbidity of impaired cognitive function.

Affective disorders include, for example, anxiety disorders (e.g., generalized anxiety disorder (GAD), social anxiety disorder (SAD; alternatively known as social phobia), panic disorder (with or without agoraphobia), posttraumatic stress disorder (PTSD), obsessive-compulsive disorder (OCD), separation anxiety disorder), mood disorders (e.g., depressive disorder, bipolar disorder) psychotic disorders (e.g., schizophrenia, schizoaffective disorder, delusional disorder), substance-related disorders (e.g., substance abuse, substance-induced disorder, substance withdrawal), cognitive disorders (e.g., dementia, delirium, Alzheimer's type dementia), affective disorder associated with neurological medical disorders (e.g., a seizure disorder, epilepsy), and affective disorders of childhood (e.g., attention disorder, attention deficit hyperactivity disorder, learning disorder, separation anxiety).

The brain is constantly integrating internal and external sensory input with memories and emotions to achieve normal thought and behavior. All of the affective disorders mentioned herein have a dysfunction in this integration and much evidence points to the amygdala as a critical brain region for this integrative process. The amygdala is a small almond-shaped mass of gray matter in the anterior extremity of the temporal lobe, and is wired to receive and integrate sensory input from external (e.g., sight or sound) and internal (e.g., tactile or memory) sources and to modify the electrophysiological output to facilitate an appropriate response. Sometimes that response is to remember that event for a lifetime, sometimes the response is to startle, and sometimes the response is to ignore the input. The neural circuit in the amygdala includes a feedback loop that can appropriately modulate memory, emotion, action, and cognition to allows people to relate and perform in their environment. Secretin can activate gene expression in the amygdala, and may alter the response of this critical region in a way to influence a core facet of different affective disorders.

Generalized Anxiety Disorder

GAD is defined as an abnormal and elevated level of anxiety experienced on a daily basis. This disorder affects about 4 million American adults and about twice as many women as men. Individuals with GAD seem unable to relax, and they may startle more easily than other people. They tend to have trouble concentrating, as well as falling or staying asleep. Unlike those with other anxiety disorders, people with GAD do not avoid certain situations as a result of their disorder. GAD is diagnosed when somebody spends at least 6 months worrying excessively about particular everyday problems. This disorder is usually accompanied by another anxiety disorder, depression, or substance abuse. GAD is currently treated with azaspirones, benzodiazepines, SSRIs, and other antidepressants.

An extensive list of symptoms and diagnostic criteria for GAD are set out in the DSM-IV §300.02 (pages 435-436).

Social Anxiety Disorder (or Social Phobia)

SAD involves overwhelming anxiety and excessive consciousness in everyday social situations. This disorder affects about 5.3 million adult Americans, and it usually begins in childhood or early adolescence. Physical symptoms often accompany the intense anxiety of social phobia, and they can include blushing, profuse, sweating, trembling, nausea, and difficulty talking. SAD often co-occurs alongside other anxiety disorders and depression. Current treatments can include anticonvulsants, benzodiazepines, beta blockers, MAOIs, SSRIs, and other antidepressants.

An extensive list of symptoms and diagnostic criteria for SAD are set out in the DSM-IV §300.23 (pages 416-417).

Panic Disorder

A panic disorder causes a person to have a feeling of terror that strikes suddenly, repeatedly, and without warning. Panic disorders affect about 2.4 million adult Americans, and they are twice as common in women as in men. Panic attacks cannot be predicted and can even occur during sleep. The intensity of a panic attack usually peaks within 10 minutes of its onset. People with panic attacks develop intense anxiety between attacks. Panic attacks are associated with increased heartbeat, sweating, fainting, nausea, chest pain, and dizziness. Depression, drug abuse, or alcoholism often co-occur in people with panic disorder. Panic disorders are currently treated with benzodiazepines, MAOIs, SSRIs, TCAs, and other antidepressants, such as DESYREL™ and EFFEXOR™.

An extensive list of symptoms and diagnostic criteria for panic disorder are set out in the DSM-IV §301.01 (pages 395 and 402).

Posttraumatic Stress Disorder

PTSD is a traumatic condition that can develop following a terrifying event. PTSD was first widely diagnosed among war veterans, but it can result from a wide variety of traumatic events, including violent attacks, such as mugging, rape, torture; kidnapping; child abuse, serious accidents such as car wrecks, and natural disasters, such as floods and earthquakes. The disorder affects about 5.2 million adult Americans. People diagnosed with PTSD have persistent frightening thoughts and memories of their ordeal, and they feel emotionally numb, especially with people to whom they once felt close. The trauma is repeatedly relived in the form of nightmares and disturbing recollections during the day. People with PTSD often experience sleep problems and are easily startled. PTST often co-occurs with depression, substance abuse, or one or more anxiety disorders. In severe cases, a person with PTSD may have trouble working or socializing with other people. Symptoms usually begin within 3 months of the trauma, but may also take years before manifestation. PTSD is only diagnosed if the symptoms last more than a month. Some people recover within 6 months, while the condition is chronic within other people. Current treatments for PTSD include MAOIs and TCAs.

An extensive list of symptoms and diagnostic criteria for PTSD are set out in the DSM-IV §309.81 (pages 427-429).

Obsessive-Compulsive Disorder

Obsessive-compulsive disorder (OCD) involves anxious thoughts or rituals that cannot be controlled by the person in whom they occur. OCD affects about 3.3 million American adults. The disorder usually first appears in childhood, adolescence, or early adulthood. Symptoms can include disturbing thoughts or images (obsessions) and performance of rituals to prevent these obsessions (compulsion). These activities consume at least one hour a day, are very distressing, and interfere with daily life. OCD often co-occurs with depression or other anxiety disorders. Currently, OCD is treated with SSRIs, TCAs, and other antidepressants.

An extensive list of symptoms and diagnostic criteria for OCD are set out in the DSM-IV §300.3 (pages 422-423) and §301.4 (and 672-673).

Other Anxiety Disorders

Other anxiety disorders can also be treated with secretin and secretin analogs. These include acute stress disorder (symptoms and diagnostic criteria in DSM-IV §308.3 (pages 431-432)), separation anxiety disorder (symptoms and diagnostic criteria in the DSM-IV §309.21 (page 113)), anxiety disorder due to a general medical condition (symptoms and diagnostic criteria in the DSM-IV §293.89 (pages 439)), substance-induced anxiety disorder (symptoms and diagnostic criteria in the DSM-IV §293.89 (pages 443-444)), panic disorder with agoraphobia (symptoms and diagnostic criteria in the DSM-IV §300.21 (pages 402-403)), adjustment disorder with anxiety (symptoms and diagnostic criteria for adjustment disorder with anxiety are set out in the DSM-IV §309.21 (pages 626-627)), anxiety disorder NOS (symptoms and diagnostic criteria in the DSM-IV §300.00 (page 404)), and specific phobias (symptoms and diagnostic criteria in the DSM-IV §300.29 (pages 410-411)).

Characteristics of Affective Disorders That Include Symptoms of Anxiety The invention includes the use of secretin and secretin analogs to treat affective disorders whose attendant symptoms include anxiety. These disorders include Alzheimer's disease, depression, bipolar disorder, schizophrenia, epilepsy, ADD, ADHD, and those mood disorders and psychotic disorders associated with symptoms of anxiety.

Alzheimer's Disease

Alzheimer's disease is the most common cause of dementia in the elderly, rendering patients dysfunctional due to cognitive impairments. Many patients with Alzheimer's show neuropsychiatric symptoms such as apathy, delusions, anxiety, and autonomic changes. Pathological hallmarks of Alzheimer's include neurofibrillary tangles (NFTs) and extracellular plaques.

An extensive list of symptoms and diagnostic criteria for Alzheimer's disease are set out in the DSM-IV §290 (pages 142-143).

Depression

Depression is a serious and widespread mental illness. In contrast to the normal emotional experiences of sadness, loss, or passing mood states, depression is persistent and can interfere significantly with an individual's ability to function. Symptoms of depression include persistent sad mood, loss of interest or pleasure in activities that were once enjoyed, change in appetite or weight, difficulty sleeping or oversleeping, physical slowing or agitation, energy loss, feelings of worthlessness or inappropriate guilt, difficulty thinking or concentrating, and recurrent thoughts of death or suicide. A diagnosis of unipolar major depression (or major depressive disorder) is made if a person has five or more of these symptoms, as well as impairment in usual functioning almost every day over the same two-week period of time.

Major depression often begins between ages 15 and 30, and sometimes even earlier. An estimated 5.3 percent of American adults from age 18 to 54 suffer from unipolar depression annually.

Some people have a chronic, but less severe, form of depression, called dysthymia (or dysthymic disorder or minor depressive disorder), that is diagnosed when the depressed mood persists for at least two years and is accompanied by at least two other symptoms of depression. An estimated 1.6% of American adults from age 18 to 54 have dysthymia annually. Many people with dysthymia also have major depressive episodes. While unipolar major depression and dysthymia are the primary forms of depression, a variety of other subtypes exist. Antidepressant medications are widely used to treat depression.

Antidepressant drugs are known to influence the functioning of certain monoamine neurotransmitters, primarily serotonin, norepinephrine, and dopamine. Older medications, such as tricyclic antidepressants (TCAs) and monoamine oxidase inhibitors (MAOIs), affect the activity of all these neurotransmitters simultaneously. However, these medications can be difficult to tolerate due to side effects or, in the case of MAOIs, dietary and medication restrictions. Newer medications, such as selective serotonin reuptake inhibitors (SSRIs), also have side-effects, though fewer. Both generations of medications yield idiosyncratic responses in particular patients, who often respond to one type of drug, but not to another.

An extensive list of symptoms and diagnostic criteria for depression/dysthymia are set out in the DSM-IV §296.2x (page 344), 296.3x (page 345), and 300.3 (page 349).

Bipolar Disorder

Bipolar disorder, also known as manic-depressive illness, is a serious brain disease that causes extreme shifts in mood, energy, and functioning. Men and women are equally likely to develop this disabling illness, which affects approximately 1% of American adults from age 18 to 54 annually.

Symptoms fall into several major categories: (1) episodes of depression, including a persistent sad mood; loss of interest or pleasure in activities that were once enjoyed, change in appetite or weight, difficulty sleeping or oversleeping, physical slowing or agitation, energy loss, feelings of worthlessness or inappropriate guilt, difficulty thinking or concentrating, and recurrent thoughts of death or suicide, (2) episodes of mania, including abnormally and persistently-elevated (high) mood or irritability accompanied by at least three of the symptoms of overly inflated self-esteem, decreased need for sleep, increased talkativeness, racing thoughts, distractibility, goal-directed activity done to excess, such as spending money, physical agitation, and excessive involvement in risky behaviors or activities, and (3) episodes of hypomania, or mild mania, including such symptoms as increased energy, elevated mood, irritability, and intrusiveness, which may cause little impairment in functioning, but are noticeable to others.

Severe depression or mania is often accompanied by periods of psychosis. Psychotic symptoms include hallucinations (hearing, seeing, or otherwise sensing the presence of stimuli that are not there) and delusions (false personal belief that are not subject to reason or contradictory evidence, and that are not explained by a person's cultural beliefs). Psychotic symptoms associated with bipolar disorder typically reflect the extreme mood state at the time they are manifested.

It is even possible for a “mixed” state to exist, in which symptoms of mania and depression are present simultaneously. Symptoms frequently include agitation, trouble sleeping, significant change in appetite, psychosis, and suicidal thinking. Depressed mood accompanies manic activation.

Episodes of mania, depression, or mixed state typically recur and become more frequent across the life span. These episodes, especially early in the course of illness, are separated by periods of normalcy during which a person suffers few to no symptoms. When four or more episodes of illness occur within a 12 month period, a person is considered to have bipolar disorder with rapid cycling. Bipolar disorder is often complicated by co-occurring alcohol or substance abuse.

Current treatments involve lithium, anticonvulsant medications, particularly valproate and carbamazepine. Newer anticonvulsant medications, including lamotrigine and gabapentin, are being studied to determine their efficacy as mood stabilizers in bipolar disorder. It is believed that anticonvulsant drugs work because they have an effect on kindling, a process in which the brain becomes increasingly sensitive to stress and eventually begins to show episodes of abnormal activity even in the absence of a stressor. It is also believed that lithium acts to block the early stages of this kindling process, and that carbamazepine and valproate act later in the progression of symptoms. During a depressive episode, people with bipolar disorder commonly require treatment with antidepressant medication. The relative efficacy of various antidepressant medications in this disorder remains uncertain. Typically, lithium or anticonvulsant mood stabilizers are given in conjunction with an antidepressant to protect against a switch into mania or rapid cycling, which can be provoked in some people with bipolar disorder by antidepressant medications. In some cases, the newer atypical antipsychotic drugs such as clozapine or olanzapine have been used to relieve severe refractory symptoms of bipolar disorder and prevent recurrences of mania. The benzodiazepines clonazepam and lorazepam can be helpful adjuncts for insomnia. Because manic-depressive illness is recurrent, long-term preventive (prophylactic) treatment is highly recommended and almost always indicated.

An extensive list of symptoms and diagnostic criteria for bipolar disorder are set out in the DSM-IV §296 (page 355-358 and 362-363).

Schizophrenia

Schizophrenia is a chronic, severe, and disabling brain disease. Approximately 1% of Americans develop schizophrenia during their lifetime, and more than 2 million Americans suffer from the illness annually.

People with schizophrenia suffer terrifying symptoms, such as hearing internal voices not heard by others, or believing that other people are reading their minds, controlling their thoughts, or plotting to harm them. These symptoms may leave a person in whom they occur, anxious, fearful, and socially withdrawn. Speech and behavior can be so disorganized that they may be incomprehensible or frightening to others.

Schizophrenia can be difficult to diagnose. Some people with symptoms of schizophrenia exhibit prolonged extremes of elated (manic) or depressed mood, and it is important to confirm that such a patient has schizophrenia or suffers for manic-depressive illness. People whose symptoms cannot be clearly categorized are sometimes diagnosed as having “schizoaffective disorder.”

The psychotic symptoms of schizophrenia used to diagnose the disorder are distorted perceptions of reality (leading individuals with schizophrenia to feel frightened, anxious, and confused), and hallucinations and illusions. Hallucinations can occur in any sensory form, including auditory, visual, tactile, gustatory, and olfactory. Schizophrenics often suffer from delusions of persecution, harassment, being cheated, poisoned, or conspired against. In addition, delusions of grandeur may occur, leading a person believe they are famous or important. A person with schizophrenia may not show the signs of normal emotion, may speak in a monotonous voice, have diminished facial expressions, and appear extremely apathetic.

Current treatments can relieve many symptoms, but most people with schizophrenia continue to suffer some symptoms throughout their lives. It has been estimated that no more than one in five individuals recovers completely from this disorder. The majority of schizophrenia patients are currently treated with antipsychotic drugs, and, in particular, the atypical antipsychotic, clozapine. Newer antipsychotics such as risperidone and olanzapine are also used because they are better tolerated despite the fact they may not be as effective. Antipsychotics may be effective in treating hallucinations and delusions but not with other symptoms such as reduced motivation and emotional expressiveness. Depression may co-occur with schizophrenia, and when a schizophrenic also suffers from depression, the symptoms of schizophrenia may also worsen. When people with schizophrenia become depressed, other symptoms may worsen, requiring the additional administration of an antidepressant.

An extensive list of symptoms and diagnostic criteria for schizophrenia are set out in the DSM-IV §295 (pages 285-286).

Epilepsy

Epilepsy is a brain disorder in which clusters of nerve cells in the brain sometimes signal abnormally, leading to seizures that, in turn, causes strange sensations, emotions, and behaviors, or even convulsions, muscle spasms, and loss of consciousness. More than 2 million people in the United States have experienced an unprovoked seizure, or have been diagnosed with epilepsy.

Only when a person has had two or more seizures is a diagnosis of epilepsy made. There are at least 30 different types of seizures, and hundreds of different epilepsy syndromes. Each epilepsy syndrome is defined by specific symptoms (for example, automatisms, auras) that include seizure.

Different tests are used to determine whether a person has epilepsy and, if so, what kind of seizures the person has. Theses tests include EEG monitoring with or without video monitoring, brain scans (CT, computed tomography; PET, positron emission tomography; MRI, magnetic resonance imaging), medical history, blood test (to screen for metabolic or genetic disorders associated with seizures; or presence of infection, poisoning, anemia, and diabetes that may cause seizures). Accurate diagnosis of epilepsy is crucial for selecting an effective treatment.

Currently available treatments can control seizures at least some of the time in 80% of people with epilepsy. However, 20% (600,000 people) have intractable seizures, while another 10% feel they get inadequate relief from available treatment. Antiepileptic drugs are commonly used. More than 20 such drugs are available, with different benefits and side effects. The most often prescribed antiepileptics are carbamazapine, valproate and phenytoin. For absence seizure, ethosuximide is often the primary treatment. Newer drugs include tiagabine, lamotrigine, gabapentin, topiramate, levetiracetam, felbamate, zonisamide and oxcarbazapine. Patients with stereotyped recurrent seizures are often treated with diazepam formulated as a gel administered rectally to prevent prolonged seizures and status epilepticus. Surgical procedures, such as removal of a seizure focus (temporal lobe resection), are sometimes performed when seizures cannot be controlled by medication. A vagus nerve stimulator may be used in people with seizures not well controlled by medication. The stimulator is a battery-powered device surgically implanted under the skin of the chest, and is attached to the vagus nerve in the lower neck. The device delivers short burst of electrical energy to the brain via the vagus. On average, the stimulator reduces seizure by about 20-40 percent.

Attention-Deficit Disorder With or Without Hyperactivity Disorder

Attention-deficit disorder (ADD), with or without hyperactivity disorder (ADHD), is a prevalent condition characterized by impaired attention, excessive motor activity, and impulsivity compared to that observed in normal individuals at a comparable level of development.

Some hyperactive-impulsive or inattentive symptoms that cause impairment must have been present before age 7 years for ADHD to be diagnosed, although diagnosis may occur several years after symptoms were first present. Some impairment from the symptoms must be present in at least two settings (i.e., at home and at school or work).

Attention-deficit manifests itself as a lack of close attention to details and often careless mistakes in such tasks as schoolwork. Individuals with ADHD are easily distracted and frequently interrupt ongoing tasks. Hyperactivity may manifest itself as fidgeting or squirming, or by not remaining seated when expected to do so. In adolescents and adults, hyperactivity takes the form of feelings of restlessness and difficulty in engaging in quiet sedentary activities. Impulsiveness appears to result from an inability to curb immediate reactions or to think before acting. The prevalence of ADHD is estimated at 3-5% in school-age children. Mood and anxiety disorders are more prevalent in individuals with ADHD.

Currently, ADHD is treated with psychostimulant medications, including methylphenidate (e.g., RITALIN™) and amphetamines (e.g., DEXEDRINE™). Antidepressants are prescribed for children who show poor response to stimulants, who have unacceptable side-effects, or who additionally have mood disorders, anxiety disorders, or both. Tricyclic antidrepressant have been demonstrated to be clinically efficacious in 60-70% of children with ADHD.

An extensive list of symptoms and diagnostic criteria for ADD and ADHD are set out in the DSM-IV §314 (pages 83-85).

Mood Disorders Mood disorders are often accompanied by symptoms of anxiety. Such mood disorders include mood disorder due to a general medical condition, substance-induced mood disorder, bipolar I disorder, schizoaffective disorder (bipolar type), bipolar disorder NOS, bipolar II disorder, cyclothymic disorder, major depressive disorder, schizoaffective disorder (depressive type), depressive disorder NOS, dysthymic disorder, and adjustment disorder with depressed mood. These disorders are further detailed in the DSM-IV.

Psychotic Disorders

Psychotic disorders are often accompanied by symptoms of anxiety. Such mood disorders include psychotic disorder due to a general medical condition, substance-induced psychotic disorder, schizophrenia, schizophreniform disorder, schizoaffective disorder, mood disorder with schichotic features, delusional disorder, psychotic disorder NOS, and brief psychotic disorder. These disorders are further detailed in the DSM-IV.

Mental Disorders

Mental disorders are often accompanied by symptoms of anxiety. Such mental disorders include delirium due to a general medical condition, delirium due to multiple etiologies, dementia due to multiple etiologies, vascular dementia, dementia due to a general medical condition, dementia of the Alzheimer's type, dementia NOS, amnestic disorder due to general medical condition, sexual dysfunction, sleep disorders, eating disorders, and personality change. These disorders are further detailed in the DSM-IV.

Alcohol-Withdrawal and Other Substance of Abuse-Withdrawal

The substance-specific syndrome associated with withdrawal causes significant distress or impairment in social, occupational, or other important areas of functioning. Anxiety is a frequent symptom in alcohol-withdrawal and other substance of abuse-withdrawal. An extensive list of symptoms and diagnostic criteria are set out in the DSM-IV. This include symptoms and diagnostic criteria for alcohol-withdrawal (DSM-IV §291.8, pages 198-199), amphetamine-withdrawal (DSM-IV §292.0, page 209), cocaine-withdrawal (DSM-IV §292.0, page 225), nicotine-withdrawal (DSM-IV §292.0, page 245), and opioid-withdrawal (DSM-IV §292.0, page 251), among others.

Secretin and secretin analogs can be used to treat symptoms of these withdrawal conditions, including symptoms of anxiety.

Eating Disorders

Eating disorders are characterized by severe disturbances in eating behavior, and are often accompanied by symptoms of anxiety. Eating disorders can occur in association with an anxiety or mood disorder and often meet criteria for major depressive disorder. A list of symptoms and diagnostic criteria for anorexia nervosa, bulimia nervosa, and eating disorder NOS are set out in the DSM-IV at §307.1 (pages 544-545), §307.51 (pages 549-550), and §307.50 (page 550).

Secretin and secretin analogs can be used to treat symptoms of these eating disorders, including symptoms of anxiety.

Sexual Disorders

Sexual dysfunctions that affect males or females often have a component of anxiety associated with sexual contacts. Sexual dysfunctions can be persistent or recurrent, and are often associated with marked distress and interpersonal difficulties. Fear and anxiety about embarrassment resulting from these disorders are often components of resulting sexual dysfunction. Sexual disorders can include such sexual dysfunctions as premature ejaculation, erectile dysfunction, or inability to achieve orgasm. Sexual dysfunctions can occur in association with an anxiety or mood disorder. These disorders are further detailed in the DSM-IV (pages 493-522).

Secretin and secretin analogs can be used to treat symptoms of these sexual disorders, including symptoms of anxiety.

Standard Animal Models of Affective Disorders

Animal models of affective disorders provide a setting for defining mechanisms of pathology and examining new therapeutic approaches. Various experimental models have been used for over a decade to help understand the biological processes and to identify potential treatments for these disorders in which a common symptom is anxiety. For example, both fear potentiated startle and pre-pulse inhibition are phenomena that can be demonstrated in man and manipulated in animal studies to model abnormal amygdala function. These models represent predictive settings for potential clinical efficacy to treat affective disorders as described herein.

Fear-Potentiated Startle

The fear-potentiated startle paradigm, that is, increased startle in the presence of a conditioned fear stimulus (CFS), is a learned fear paradigm that has been shown to involve the central amygdala (see, e.g., Davis, Behav. Neurosci., 100:814-824, 1986). Fear potentiated startle evokes a neurological process that mimics the behavioral pathology manifest in post traumatic stress disorder and other anxiety-based diseases. Elevated anxiety impairs sensory processing with consequent deterioration of memory, cognition, and social function. Anxiogenic states seen in human conditions such as generalized social phobia (Stein et al., Arch. Gen. Psychiatry, 59:1027-1034, 2002) or drug-induced animal models of anxiety (Sanders and Shekhar, Pharmacol. Biochem. Behav., 52:701-706, 1995) are accompanied by abnormal amygdala function.

Compounds clinically useful in treating anxiety are effective in these models, indicating that testing potential therapies for affective disorders in these models is predictive of clinical efficacy.

The fear-potentiated startle paradigm performed in rats has become increasingly popular as a tool for evaluating the potential efficacy of putative anxiolytic (anxiety-reducing) compounds because of its reported sensitivity to anxiolytic drugs (Helton et al., J. Pharmacol. Exp. Ther, 284:651-660, 1998; Davis, Psychopharmacology (Berl) 62:1-7, 1979; Davis, Behav. Neurosci., 100:814-824, 1986). In this paradigm the amplitude of the acoustic startle is reliably enhanced when elicited in the presence of cues previously paired with shock. This potentiated response to fear is often described as a surrogate state of pathological anxiety.

Previous human studies have demonstrated that both the baseline and fear-potentiated responses can be inhibited by anxiolytic drugs, such as the benzodiazepine alprazolam (Riba et al., Psychopharmacology (Berl), 157:358-367, 2001). Measures of fear-potentiated startle response in rats and humans provide a good indication for the potential anxiolytic activity of a drug (for example, Belzung, Current Opinion in Investigational Drugs. 2(8):1108-1111, 2001; Nestler et al., Neuron, 34:13-25, 2002). Thus, these known models can be used to confirm the efficacy of secretin and secretin analogs as therapies for affective disorders.

Pre-Pulse Inhibition

Pre-pulse inhibition (PPI) is a phenomenon in which a weak sensory pre-pulse reduces the startle normally induced by a closely following strong startle stimulus. This phenomena is not learned and is thought to demonstrate the sensory motor gating that serves to avoid sensory interference and allow independent processing of discrete stimuli. Impairment of PPI thus leads to sensory confusion (Braff and Geyer, Arch. Gen. Psychiatry, 47:181-188, 1990)(Perry and Braff, Am. J. Psychiatry, 151:363-367, 1994).

Impairment of PPI is observed in diseases that manifest sensory processing dysfunction (SPD) such as schizophrenia (Perry et al., Arch. Gen. Psychiatry, 56:277-281, 1999), Huntington's disease (Swerdlow et al., J .Neurol. Neurosurg. Psychiatry, 58:192-200, 1995), ADHD (Ornitz et al., Psychophysiology, 29:437-451, 1992), OCD (Swerdlow et al., Biol. Psychiatry, 33:298-301, 1993), and Tourette's syndrome (Castellanos et al., Biol. Psychiatry, 39:33-41, 1996).

PPI impairment can be induced by the use of NMDA antagonists such as phencyclidine (PCP). PCP has been shown to exert its effect through action in the amygdala and hippocampus (Bakshi and Geyer, J. Neurosci., 18:8394-8401, 1998) probably by consequent elevation of noradrenaline (Bakshi and Geyer, J. Pharmacol. Exp. Ther., 283:666-674, 1997). Atypical anti-psychotic medications such as Clozapine and Olanzapine reverse the PPI impairment induced by phencyclidine (Le Pen and Moreau, Neuropsychopharmacology, 27:1-11, 2002). PCP-induced disruption of PPI therefore provides a setting of neuropathology of the amygdala as it has been defined in many clinical presentations of sensory processing dysfunction, and reversal of this impairment by drugs is predictive of efficacy in human conditions with sensory processing deficits, such as the affective disorders described herein.

Secretin and Secretin Analogs

The invention in base, at least in part, on the discovery that secretin and secretin analogs can be used to treat affective disorders and other disorders that include attendant anxiety symptoms, as well as to enhance cognition, including learning and memory, and to reduce fear-potentiated startle response and to reduce pre-pulse inhibition response.

The amino acid sequence of secretin has long been known. The invention includes use of human secretin, as well as homologous secretins from mammals including bovine, porcine, equine, canine, and other mammalian secretins that have the same or essentially the same biological activity in the fear-potentiated startle and PPI models described herein.

The invention also includes the use of polypeptides that have a sequence substantially identical to secretin, as long as they have at least 50% of the biological activity of naturally occurring human or porcine secretin in the animal models described herein. A polypeptide which is “substantially identical” to a given reference polypeptide is a polypeptide having a sequence that has at least 85% identity to the sequence of the given reference polypeptide sequence. Substantially identical polypeptides can also have a higher percentage identity, e.g., 90%, 95%, 98%, or 99%. Identity or substantial identity can easily be determined by eye or computer on this relatively short polypeptide. A given polypeptide is 100% identical to naturally occurring human secretin when all amino acids are the same in both polypeptides, and, for example, is 50% identical, when half of the amino acids in the respective sequences have a one-to-one correspondence (i.e., half of the amino acid locations in the given sequence are identical to the amino acid locations in the naturally occurring sequence).

The invention also encompasses polypeptides that are functionally equivalent to secretin. These polypeptides are equivalent to secretin in that they are capable of carrying out one or more of the functions of secretin in the animal models of fear-potentiated startle or PPI described herein. Such polypeptides have at least 50%, or 60%, 75%, 80%, or even 90% of one or more of the biological activities of full-length secretin. Such comparisons are generally based on an assay of biological activity in which equal concentrations of the polypeptides are used and compared. The comparison can also be based on the amount of the polypeptide required to reach 50% of the maximal stimulation obtainable.

Functionally equivalent polypeptides can be those, for example, that contain additional or substituted amino acid residues compared to full-length, wild-type (naturally occurring) human secretin. Substitutions may be made on the basis of similarity in polarity, charge, solubility, hydrophobicity, hydrophilicity, and/or the amphipathic nature of the residues involved. For example, a functionally equivalent polypeptide is one in which 10% or fewer of the amino acids full-length, naturally occurring human secretin are replaced by conservative amino acid substitutions, and the functionally equivalent polypeptide maintains at least 50% of the biological activity of full-length secretin.

Amino acid substitution refers to the substitution of one amino acid for another amino acid of the same class. These substitutions can include amino acid residues that represent either a conservative or non-conservative change (or, where more than one residue is varied, possibly both). A “conservative” substitution is one in which one amino acid residue is replaced with another having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The invention includes polypeptides that include one, two, three, five, or more conservative amino acid substitutions, where the resulting mutant polypeptide has at least one biological activity that is the same, or substantially the same, as a biological activity of wildtype secretin.

Polypeptides that are functionally equivalent to human full-length, naturally occurring secretin can be made using random mutagenesis on the encoding nucleic acids by techniques well known to those skilled in the art. It is more likely, however, that such polypeptides will be generated by site-directed mutagenesis (again using techniques well known to those skilled in the art). These polypeptides may have increased functionality or decreased functionality.

To design functionally equivalent polypeptides, it is useful to distinguish between conserved positions and variable positions. This can be done by aligning the amino acid sequence of a protein of the invention from one species with its homolog from another species. Skilled artisans will recognize that conserved amino acid residues are more likely to be necessary for preservation of function. Thus, it is preferable that conserved residues are not altered.

Mutations within the coding sequence of a nucleic acid molecule encoding secretin can be made to generate variant genes that are better suited for expression in a selected host cell. For example, N-linked glycosylation sites can be altered or eliminated to achieve, for example, expression of a homogeneous product that is more easily recovered and purified from yeast hosts that are known to hyperglycosylate N-linked sites. To this end, a variety of amino acid substitutions at one or both of the first or third amino acid positions of any one or more of the glycosylation recognition sequences which occur, and/or an amino acid deletion at the second position of any one or more of such recognition sequences, will prevent glycosylation at the modified tripeptide sequence (see, for example, Miyajima et al., EMBO J., 5:1193, 1986).

The polypeptide secretin analogs used as part of the invention can be expressed fused to another polypeptide, for example, a marker polypeptide or fusion partner. For example, the polypeptide can be fused to a hexa-histidine tag to facilitate purification of bacterially expressed protein or a hemagglutinin tag to facilitate purification of protein expressed in eukaryotic cells.

A fusion protein may be readily purified by utilizing an antibody specific for the fusion protein being expressed. For example, a system described by Janknecht et al. allows for the ready purification of non-denatured fusion proteins expressed in human cell lines (Proc. Natl. Acad. Sci. USA 88:8972-8976, 1991). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni2+nitriloacetic acid-agarose columns and histidine-tagged proteins are selectively eluted with imidazole-containing buffers.

The polypeptide secretin analogs used as part of the invention can be chemically synthesized (for example, see Creighton, “Proteins: Structures and Molecular Principles,” W. H. Freeman & Co., NY, 1983), or, perhaps more advantageously, produced by recombinant DNA technology as described herein. For additional guidance, skilled artisans may consult Ausubel et al. (supra), Sambrook et al. (“Molecular Cloning, A Laboratory Manual,” Cold Spring Harbor Press, Cold Spring Harbor, N.Y., 1989), and, particularly for examples of chemical synthesis Gait, M. J. Ed. (“Oligonucleotide Synthesis,” IRL Press, Oxford, 1984).

Secretin and Secretin Analogs Reduce Anxiety Without Adverse Effect on Cognition and Locomotor Activity

Drugs with anxiolytic or anti-depression activity often demonstrate unwanted side-effects, including, but not limited to, sedation, amnesia or other cognitive impairment, hyperactivity or hypoactivity. A standard test for these unwanted side-effects is to quantify activity and emotionality to a novel surrounding in rats after repeated exposure to drug. An additional test to measure effects of a drug on aspects of learning and memory is represented by the M Swim Maze.

The invention is based, at least in part, on the discovery that secretin and secretin analogs are effective anxiolytics, but do not cause adverse effects on cognition (including learning and memory) or locomotory function. In fact, secretin and secretin analogs enhance learning and memory.

Spatial Navigation in an M Swim Maze

The M swim maze was developed to test spatial learning and memory (e.g., functional memory). The animal has no visual or spatial cues in the pool and must rely on extra-maze cues (e.g., light setup outside the pool that can be seen by the swimming animal). Through a series of trials a rat develops “place learning” or knowledge about the position of the escape platform based upon the extra-maze cues. The platform can be moved to a different arm of the M configuration each day, combining spatial memory with working memory. This paradigm involves extinction of the prior memory and resolution of a new spatial problem. Many drugs that have anxiolytic or anti-depressive effects have detrimental effects on functional memory important for daily life. Additionally, spatial learning and memory tasks in rodents during stressful activities, such as escape from water, are useful to evaluate drugs for unwanted side effects of impairment of functional memory.

Results in rodents correlate well to those in humans and other mammals.

Therefore, decreased performance in this model would indicate a negative locomotor or cognitive side-effect of drug treatment. An improvement may indicate improved cognition due to reduced stress or anxiety from task performance.

The invention is based, at least in part, on the discovery that, over a wide range of dosages, rats injected with secretin significantly improved their learning and cognition in the M swim maze without impairment of locomotory performance. These results are predictive of similar improvements in learning and memory in other mammals, including humans, upon administration of secretin and secretin analogs.

Use of Secretin and Secretin Analogs Reduced Anxiety and Enhanced Cognition

Patients to whom secretin and secretin analogs are to be administered will tend to present with symptoms of anxiety, fear, avoidance, increased arousal, and/or be depressed, elevated, expansive, or irritable, and/or have delusions, hallucinations, disorganized speech, or grossly disorganized behavior. The affective disorders described herein, such as anxiety disorders, mood disorders, or psychotic disorders, can be diagnosed using the DSM-IV criteria.

Diagnosis

The DSM-IV criteria can be used to diagnose the affective disorders described herein (such as depression, generalized anxiety disorder, panic disorder, obsessive-compulsive disorder, post-traumatic stress disorder, social anxiety disorder, anxiety disorder NOS). Detailed classification of anxiety disorders is provided in the DSM-IV. It is important to recognize that there are alternative nomenclatures, nosologies, and classification systems for pathologic psychological conditions, and that these systems evolve with medical scientific progress. Consequently, methods of diagnosing disorders currently described in the DSM-IV can be expected to evolve over time to encompass new medical knowledge. The invention includes the use of secretin and secretin analogs to treat disorders currently specified in the DSM-IV as well as anxiety disorders as specified in the future.

Measures of Clinical Efficacy

Clinical improvement in symptoms of affective disorders may be reflected in reduced anxiety or in improvement of symptoms other than anxiety. Depression, anxiety, schizophrenia, Alzheimer's disease, and other disorders including symptoms of anxiety have established disease scales with which to monitor efficacy of drug treatment. For example, improvements in the severity of symptoms such as depression or anxiety can be assessed typically using the Hamilton Anxiety Scale, Hamilton Depression Scale, or Hamilton/Melancholia Scale. A reduction in such symptoms indicates that a patient's condition has improved. An additional example is that improvement in the symptoms of schizophrenia can be assessed using the Scales for the Assessment of Negative Symptoms (SANS), the Positive and Negative Syndrome Scale (PANSS) and/or the Global Assessment Scales (CGI). Likewise, one can measure improvement in Alzheimer's disease, as well as general cognitive improvement in learning and memory, using the Wechsler scale and/or the Wisconsin Card Sort Test.

These clinical evaluation tests are provided only as examples to illustrate some clinical assessment tools that can indicate disease improvement subsequent to secretin and secretin analogs treatment. Other sources, such as the DSM-IV, are also useful in judging improvements in the symptoms of the affective disorders described herein.

Secretin and Secretin Analog Administration

Secretin and secretin analogs for use in accordance with the present invention can be formulated in a conventional manner using one or more physiologically acceptable carriers or excipients. For example, secretin and secretin analogs and their physiologically acceptable salts and solvates can be formulated for administration by inhalation or insufflation (either through the mouth or the nose) or oral, buccal, parenteral, or rectal administration.

For oral administration, secretin and secretin analogs may take the form of, for example, tablets or capsules prepared by conventional means with pharmaceutically acceptable excipients such as binding agents (for example, pregelatinised maize starch, polyvinylpyrrolidone or hydroxypropyl methylcellulose); fillers (for example, lactose, microcrystalline cellulose or calcium hydrogen phosphate); lubricants (for example, magnesium stearate, talc or silica); disintegrants (for example, potato starch or sodium starch glycolate); or wetting agents (for example, sodium lauryl sulphate). The tablets may be coated by methods well known in the art.

Liquid preparations for oral administration may take the form of, for example, solutions, syrups, or suspensions, or they may be presented as a dry product for constitution with water or other suitable vehicle before use. Such liquid preparations may be prepared by conventional means with pharmaceutically acceptable additives such as suspending agents (for example, sorbitol syrup, cellulose derivatives or hydrogenated edible fats); emulsifying agents (for example, lecithin or acacia); non-aqueous vehicles (for example, almond oil, oily esters, ethyl alcohol or fractionated vegetable oils); and preservatives (for example, methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

For buccal administration secretin and secretin analogs may take the form of tablets or lozenges formulated in conventional manner.

For administration by inhalation, secretin and secretin analogs for use according to the present invention can be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, for example, gelatin for use in an inhaler or insufflator may be formulated containing a powder mix of secretin or a secretin analog and a suitable powder base such as lactose or starch.

Secretin and secretin analogs can be formulated for parenteral administration by injection, for example, by bolus injection or continuous infusion. Formulations for injection may be presented in unit dosage form, for example, in ampoules or in multi-dose containers, with an added preservative. The secretin and secretin analogs may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, for example, sterile pyrogen-free water, before use.

The secretin and secretin analogs can also be formulated in rectal compositions such as suppositories or retention enemas, for example, containing conventional suppository bases such as cocoa butter or other glycerides.

In addition to the formulations described previously, the secretin and secretin analogs can also be formulated as a depot preparation. Such long-acting formulations may be administered by implantation (for example subcutaneously or intramuscularly) or by intramuscular injection. Thus, for example, the secretin and secretin analogs may be formulated with suitable polymeric or hydrophobic materials (for example as an emulsion in an acceptable oil) or ion exchange resins, or as sparingly soluble derivatives, for example, as a sparingly soluble salt.

The secretin and secretin analogs may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

Secretin and secretin analogs can also contain a carrier or excipient, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. The nucleic acids, polypeptides, antibodies, or modulatory compounds of the invention can be administered by any standard route of administration. For example, administration can be parenteral, intravenous, subcutaneous, intramuscular, intracranial, intraorbital, ophthalmic, intraventricular, intracapsular, intraspinal, intracistemal, intraperitoneal, transmucosal, or oral. The modulatory compound can be formulated in various ways, according to the corresponding route of administration. For example, liquid solutions can be made for ingestion or injection; gels or powders can be made for ingestion, inhalation, or topical application. Methods for making such formulations are well known and can be found in, for example, “Remington's Pharmaceutical Sciences”.

It is recognized that the pharmaceutical compositions and methods described herein can be used independently or in combination with one another. That is, subjects can be administered one or more of the pharmaceutical compositions, subjected to one or more of the therapeutic methods described herein, or both, in temporally overlapping or non-overlapping regimens. When therapies overlap temporally, the therapies may generally occur in any order and can be simultaneous (e.g., administered simultaneously together in a composite composition or simultaneously but as separate compositions) or interspersed. Specifically, secretin and secretin analogs can be administered alone or in conjunction with other drugs. By way of example, a subject afflicted with a disorder described herein can be simultaneously or sequentially administered both secretin or a secretin analog and another anxiolytic drug. Even when coadministered with another anxiolytic drug secretin and secretin analogs can alleviate symptoms of anxiety associated with a mood or cognitive disorder by reducing the spectrum or severity of disease symptoms.

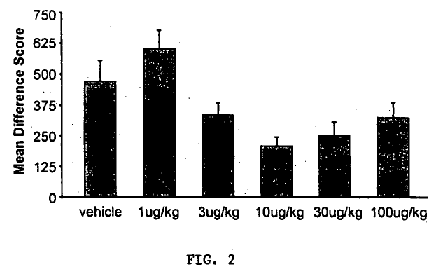

Animal tests in rats indicate that a minimum i.p. dose of 3 μg/kg secretin possesses anxiolytic properties in a fear-potentiated startle paradigm (FIG. 2). An M swim maze training and recall protocol indicates that a minimum i.v. dose of 0.4 g/kg secretin improves functional memory with significant effects also observed at 4.0 μg/kg and 40 μg/kg. A minimum i.v. or i.p dose of 4 μg/kg secretin was sufficient to detect significant changes in brain gene expression as measured by Fos protein expression. A peak change in Fos protein expression following i.p. or i.v. secretin administration was observed at 40 μg/kg.

In humans, the effective dosage levels of secretin administered i.v. to treat symptoms of anxiety disorders, other disorders associated with anxiety, or to enhance cognition, ranges from about 0.04 μg/kg to about 400 μg/kg. A typical starting dose is 0.4 μg/kg. A typical starting dose for i.v. administration is 4.0 μg/kg. Dosage of secretin and secretin analogs by means other than i.v. administration can be adjusted to take into account pharmacokinetic differences associated with different admininstration routes (e.g., subcutaneously or oral vs. i.v.). Thus, a typical starting dose for subcutaneous administration can be estimated to be 10 μg/kg. The dosage will tend to be similar for oral administration.

Typically, a patient will be treated with secretin and secretin analogs on a single occasion, once per day, once every other day, once per week, once per month, or according to another clinically appropriate schedule, depending on the severity of anxiety symptoms, response to treatment, route of secretin and secretin analogs administration, and dose of secretin or secretin analog administered.

Safety of Secretin and Secretin Analogs

The hormone secretin is a polypeptide hormone naturally secreted by the mucosa of the duodenum in normal individuals. Secretin is also a neuropeptide produced by normal brain cells. In particular, secretin and secretin receptors are expressed in mammalian brain tissue, including the cerebellum. Secretin has already been approved by the United States Food and Drug Administration as safe for use in humans as part of a diagnostic procedure for pancreatic function.

Animal Models

In general, known animal models of anxiety and depression are susceptible to the actions of anti-anxiety/anti-depressant drugs as measured during performance of a specific task or as a response to stress manifested as behavioral impairments. These tests have been effective at predicting the efficacy of new medications in reducing anxiety or depression. For example, benzodiazepines have been shown to decrease the degree to which an animal shows fear-potentiated startle. Similarly, animal models have been used to demonstrate the efficacy of medications that enhance cognition, including learning and memory.

Rodents, such as laboratory rats, are routinely used to study the therapeutic effectiveness and safety of drugs, including anxiolytic drugs and drugs that enhance cognition. Effectiveness in laboratory rats at reducing symptoms of anxiety, as well as symptoms of disorders associated with anxiety, and in enhancing cognition (e.g., learning, memory) correlates well with effectiveness in humans and other mammals.

The invention is based, at least in part, on the discovery that secretin and secretin analogs reduce symptoms of anxiety, and enhances cognition, such as learning and memory, in laboratory rats without causing adverse side-effects. Secretin and secretin analogs can be used for the same purposes in humans, as well as in other mammals, such as cattle, horses, goats, sheep, pigs, dogs, and cats.

### EXAMPLES

The invention is further described in the following examples, which do not limit the scope of the invention described in the claims. The basic animal models are described first, followed by specific examples.

Fear-Potentiated Startle Model Test Animals

A total of 72 male Sprague-Dawley rats (Charles River, Portage, Mich.) weighing 350-450 g were used. Animals were maintained on a 12:12 hour light-dark cycle (lights on at 0700) with food and water continuously available. All rats were housed in group cages of four rats each in a temperature-controlled (24° C.) animal colony.

Experimental Apparatus

Animals were trained and tested in 8×15×15 cm Plexiglas® and wire-mesh cages. Each cage floor consisted of four 6.0 mm diameter stainless-steel bars spaced 18 mm apart. Each cage was suspended between compression springs within a steel frame and located within a custom-designed 90×70×70 cm ventilated sound-attenuating chamber. Background noise (60 dB wide-band) was provided by a General Radio Type 1390-B noise generator (Concord, Mass.) and delivered through high-frequency speakers (Radio Shack Supertweeter; Tandy, Fort Worth, Tex.) located 5 cm in front of each cage. Sound level measurements (sound pressure level) were made with a Bruel & Kjaer (Marlborough, Mass.) model 2235 sound-level meter (A scale; random input) with the microphone (Type 4176) located 7 cm from the center of the speaker (approximating the distance of the rat's ear from the speaker).

Startle responses were evoked by 50 msecond, 95 dB white noise bursts (5 msecond rise-decay) generated by a Macintosh G3 computer soundfile (0-22 kHz), amplified by a Radio Shack amplifier (100 W; model MPA-200; Tandy), and delivered through the same speakers used to provide background noise. An accelerometer (model U321A02; PCB Piezotronics, Depew, N.Y.) affixed to the bottom of each cage produced a voltage output proportional to the velocity of cage movement. This output was amplified (model 483B21; PCB Piezotronics) and digitized on a scale of 0-2500 U by an InstruNET device (model 100B; GW Instruments, Somerville, Mass.) interfaced to a Macintosh G3 computer. Startle amplitude was defined as the maximal peak-to-peak voltage that occurred during the first 200 msecond after onset of the startle-eliciting stimulus.

The conditioned stimulus (CS) was a 3.7 second light (80 lux) produced by an 8 W fluorescent bulb (100 msecond rise time) located 10 cm behind each cage. Luminosity was measured using a VWR light meter (Atlanta, Ga.). The US was a 0.5 second shock, delivered to the floorbars and produced by a shock generator (SGS-004; LeHigh Valley, Beltsville, Md.). Shock intensities (measured as in Cassella et al., Physiol Behav 36:1187-91, 1986) were 0.4 mA. The presentation and sequencing of all stimuli were under the control of the Macintosh G3 computer using custom-designed software (The Experimenter; Glassbeads Inc., Newton, Conn.).

Drug Administration

Secretin (Repligen Corp., Waltham, Mass.) (1, 3, 10, 30, and 100 μg/kg) was freshly dissolved in vehicle and injected intraperitoneally immediately prior to testing. Drug doses and concentration range were selected based on those used in prior human and rat studies (for example, Goulet et al. Neuroscience 118:881-888, 2003).

Behavioral Procedures

The experiment was run in four replications. Total group sample sizes were as follows: vehicle (n=16), 1 μg/kg (n=12), 3 μg/kg (n=12), 10 μg/kg (n=12), 30 μg/kg (n=12), 100 μg/kg (n=8).

Matching. On each of 2 days, animals were placed in the test chambers and presented with 30 95 dB noise bursts at a 30 second interstimulus interval (ISI). The mean startle amplitude across the 30 stimuli on the second day was used to divide rats into groups with similar startle amplitudes.

Fear conditioning. On each of the next 2 days, rats were returned to the test chambers and 5 minutes later given the first of 10 light-footshock pairings. The 0.4 mA 0.5 second shock was delivered during the last 0.5 second of the 3.7 second light. The average intertrial interval (ITI) was 4 minutes (range, 3-5 minutes).

Testing. 24 hour after the last fear conditioning session, rats were injected i.p. with Secretin (1, 3, 10, 30, or 100 μg/kg) or vehicle and were immediately placed into the test chambers. After 5 minutes the rats received 30 95 dB noise bursts (30 second ISI) to habituate the startle response to a stable baseline prior to the test trials. Each test trial (18 total) involved the presentation of a noise burst of one of 3 intensities (95, 100, or 105 dB); half of these occurred in the presence and half in the absence of the light CS. On the CS trials the startle stimulus was presented 3.2 second after the onset of the 3.7 second light. Trial types were presented in a balanced, irregular order (30 second ITI) with the restriction that each of the 6 trial types had to occur once within each of the 3 trial blocks.

Behavioral Measurement and Data Analysis

The initial startle stimuli of the test session were used to habituate startle responses to asymptotic levels and were not included in statistical analyses. Subsequent startle responses generated by the three different startle intensities were averaged for each animal to obtain a single score for both the startle stimulus alone (baseline) and the CS and startle stimulus trials. A difference score was computed for each animal by subtracting the mean baseline startle amplitudes from the mean startle amplitudes in the presence of the CS. Analyses of variance (ANOVAs) and contrasts were computed as required.

Spatial Memory Light-Cued Water Maze Animal Model

Test Animals. A total of 40 male rats were used.

Behavioral Procedures and Data Analysis. Rats (10/group) were treated by tail vein injection with vehicle, 0.4 μg/kg/day, 4.0 μg/kg/day, and 40 μg/kg/day secretin on post natal day 25 through postnatal day 70 (45 days). Learning and memory was evaluated in a water M-maze. The evaluation consisted of 10 trials/day for each animal on 4 successive days to assess short-term memory. The goal was to escape from the maze via a platform located on the lighted arm of an M-shaped maze. After placement of the animal in the central arm of the M-shaped maze, the goal side was varied for each animal at each trial according to a predetermined computer generated sequence. The same animals were also tested 5 days after the initial testing to assess long-term memory. On that day each animal were allowed 10 trials in the maze and time to escape was measured. Significance was calculated versus the control group value for the same day of testing using a T-test.

Activity and Functional Observation Measurements

Test Animals. A total of 96 male rats and 96 female rats were used.

Behavioral Procedures and Data Analysis. Rats (24 animals/sex/group) were dosed iv with vehicle, or secretin at 0.4 μg/kg, 4.0 μg/kg, and 40 μg/kg from post natal day 25 to post natal day 70. The locomotor activity of 10 randomly selected rats/sex/group was measured on post natal day 30 (adolescent) and post natal day 72 (adult). On post natal day 30 and post natal day 72, each rat was placed in a shoebox cage equipped with the automated Photobeam Activity System (PAS; San Diego Instruments, Inc.). Locomotor activity was monitored during a 60 minute session composed of 12 5-minute intervals. The total number of photobeam breaks which occurred during each of the 12 5-minute intervals was recorded. Changes in habituation to novel environments can be assessed by comparing locomotor activity over 3 session intervals between the control versus test groups for habituation. Emotionality was recorded by following behavioral facets as described by Hall (J. Comp. Physiol. Psychol. 22:325-352, 1936) and Spyker (In: Behavioral Toxicology, Ed. B. Weiss and V. G. Laties, Plenum Press, New York, pp 311-349, 1975), including defecation, urination, rearing, grooming, and backing. A functional observation battery was performed on postnatal day 75 according to the parameters described by Irwin (Psychopharmacologia 13:222-257, 1968) to evaluate gait, posture, abnormal behavior, and vocalization.

### Example 1

### Effect of Secretin on Baseline Startle Amplitude

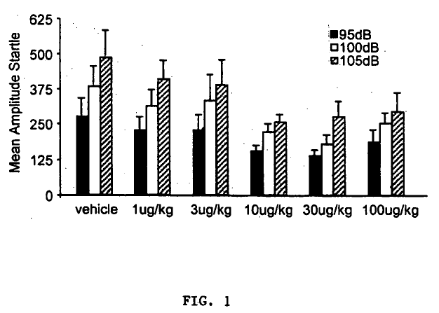

Secretin did not cause significant sensory impairment as measured by its influence on baseline startle amplitude (FIG. 1).

The interaction between group and intensity did not approach statistical significance (p>0.05). Higher doses of secretin tended to suppress baseline startle, as indicated by a trend towards lower mean startle amplitudes to the leaders and the startle stimulus alone test trials (FIG. 1) in the groups receiving 10, 30, or 100 μg/kg than in the other groups. In neither case was this trend significant, however, as there was no main effect of group in either analysis. There was a reliable main effect of startle stimulus intensity [F(2, 132)=47.525, p<0.01] and a significant linear trend [F(1, 66)=70.258; p<0.01], indicating that noise bursts of higher intensities elicited startle responses of greater amplitudes.

### Example 2

### Effect of Secretin on Expression of Fear-Potentiated Startle

FIG. 2 demonstrates that secretin dose dependently impaired expression of fear-potentiated startle when administered immediately prior to testing, with the greatest impairment occurring in the treatment group receiving 10 μg/kg. Statistical analysis confirmed these observations. A one-way ANOVA on the difference score data revealed a significant main effect of test group [F(5, 66)=2.394; p<0.05] and a significant quadratic trend [F(4, 66)=2.648; p<0.05]. Thus, secretin caused a significant reduction in startle (i.e., anxiety) in a model representing a surrogate state of pathological anxiety.

### Example 3

### Effect of Secretin on Functional Memory in a Water M-Maze

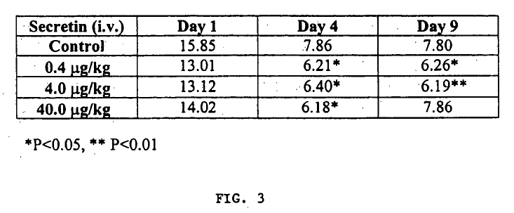

As shown in FIG. 3, rats treated with 0.4 or 4.0 μg/kg/day improved in learning performance after 4 training days relative to controls, and both dose groups maintained that improvement when the test was repeated after a 5 day rest period.

In FIG. 3, the values for each treatment group represent the average time (seconds) to complete the test. The average is taken over the 20 animals and 10 trials/day. Significance is calculated versus the control group value from the same day of testing using a T-test. The data reveal that secretin does not impair the ability to learn and remember a spatial associative memory task (light and escape). Moreover, in these rats there was an enhancement (that is, reduction) in the time to escape (Day 4; indicating enhanced learning) that persisted for at least nine days (indicating enhanced memory). This demonstrates the ability of secretin to help maintain or improve functional memory.

Thus, an anxiolytic secretin-mediated effect can by detected in a behavioral paradigm whose measure is a cognitive assessment.

### Example 4

### Effect of Secretin on Locomotor Activity and Functional Observation Measurements

Secretin did not change the response of rats to a novel environment regarding emotionality or motor activity as detected in the functional observation battery after repeat dose of secretin. No significant difference in urination, defecation, grooming, backing or rearing over 1 hour was seen in rats dosed daily for 45 days with doses of 0.4-40 μg/kg/dose. There was no evidence of induced hyper- or hypo- motor activity in adolescent or adult animals. No significant difference in horizontal, vertical or total distance moved over 1 hour was seen in rats dosed for 45 days with doses of 0.4-40 μg/kg/dose. These results demonstrate that there is no disturbance of normal behavior after repeat dosing with secretin.

### Example 5

### Treatment of OCD by Administering Secretin

Sixteen (16) outpatients with OCD, age 14-40 years, inclusive, are included in this open-label, dose-escalation study. Patients are either on treatment with a Selective Serotonin Reuptake Inhibitor (SSRI) or on no treatment. In the former case, they must have residual symptoms despite an adequate trial of at least one SSRI. In the latter case, patients may be either treatment-naïve or have stopped treatment with an SSRI at least 12 weeks before screening for this protocol.

The first 8 patients receive synthetic human secretin, formulated as a lyophilized cake from a 1 ml solution of 5 mM KPO₄, 40 mg/ml mannitol, and 2.7 mg/ml secretin, and resuspended immediately prior to use with 0.75 ml of sterile saline. Each patient receives 10 μg/kg of this resuspended secretin formulation subcutaneously (SC) three times per week for one month and are divided between those receiving SSRIs (n=4) and those not receiving SSRIs (n=4). The latter patients (those not receiving SSRIs) are divided between newly diagnosed, SSRI-naive patients (n=2) and patients who received SSRIs in the past but who have discontinued treatment (n=2). An additional 8 patients receive secretin 20 mcg/kg SC three times weekly for one month, divided as above.

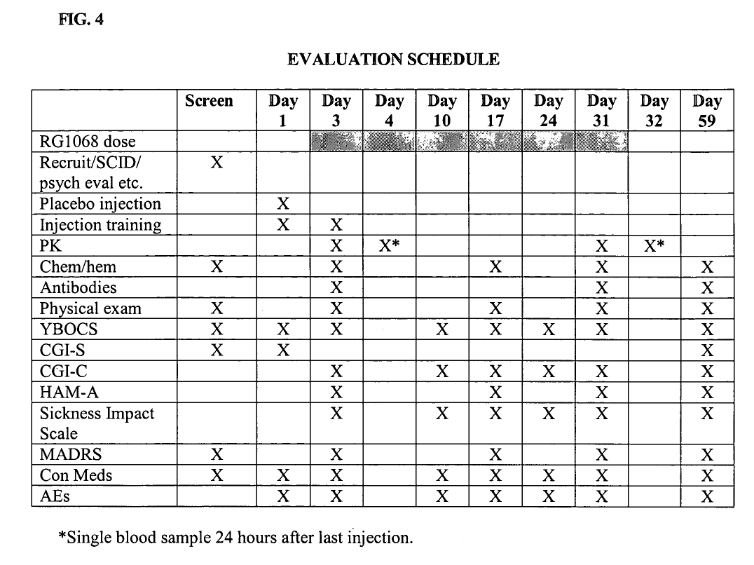

The primary efficacy outcomes are the change in the Yale-Brown Obsessive Compulsive Disorder Scale (YBOCS) and the Clinical Global Impression of Change (CGI-C) as compared to the Clinical Global Impression of Severity (CGI-C). Additional efficacy assessments include the Hamilton Anxiety Scale (HAM-A), the Montgomery-Asberg Depression Rating Scale (MADRS), and the Sickness Impact Scale. Pharmacokinetics are performed around the first and last injections in a subset of patients. Formation of any antibodies to secretin is checked. All evaluations are made according to the Evaluation Schedule in FIG. 4.

### Example 6

### Treatment of GAD by Administering Secretin

A 56 year old, 80 kg, man is treated for Generalized Anxiety Disorder (GAD). He is assessed for GAD using the diagnosis criteria and characteristics described in DSM-IV. In particular, the patient suffers from debilitating and uncontrollable anxiety and worry about his job, his family, and his future. He feels physically fatigued, he has great difficulty concentrating at work, he is often irritable around his wife and children, and he has trouble sleeping almost every night. He appears otherwise healthy, both physically and mentally.

The patient is administered secretin at a dose of 4 μg/kg/day (320 μg/day), using subcutaneous administration. The initial treatment period lasts 6 consecutive weeks. Upon improvement in the symptoms of GAD, a physician should regularly assess the patient, and administer secretin intermittently whenever the patient's GAD symptoms require it.

### Example 7

### Treatment of Schizophrenia Symptoms by Administering Secretin

A 39 year old, 50 kg, female patient is treated for positive and negative symptoms of schizophrenia. Among other symptoms, she suffers from persistent delusions and frightening hallucinations that create a permanently heightened sense of anxiety. She is diagnosed by a physician using the criteria and characteristics described in DSM-IV for schizophrenia. The patient also fulfills the criteria of primary deficit disorder, with a SANS score of greater than 40 (see Kirkpatrick et al., Psychiatry Research 30:119-123, 1989; Andreasen, Scales for the Assessment of Negative Symptoms (SANS), Iowa City, Iowa, 1983). Before treatment initiation, and at 2 week intervals following initiation of treatment, severity of the disorder is assessed by a physician using the PANSS, SANS, and CGI. The Wisconsin Card Sort Test is also used to provide a cognitive rating of the patient. This test is performed at the initiation of treatment, and again after 2, 4, and 6 weeks of treatment. The physician is able reliably to assess changes in the patient's condition by comparing the treatment results over two-week intervals. The side-effects of secretin treatment are assessed biweekly according to the UKU side-effects rating scales and other, similar scales.

Under supervision of a physician, the patient receives secretin at a dosage of 2 μg/kg/day (that is, 100 μg/day) using intravenous administration; intravenous administration is often recommended for inpatient populations, such as schizophrenics.

Upon improvement in symptoms of schizophrenia, a physician should regularly assess the patient, and administer secretin regularly to avoid recurrence of the patient's schizophrenia symptoms.

### Example 8

### Memory Enhancement in Alzheimer's Disease

A 69 year old, 70 kg, man is diagnosed by a physician as having advanced Alzheimer's Disease and exhibiting signs of anxiety (i.e., anger and frustration). The most debilitating symptom exhibited by the man is his very poor ability to form new memories. This makes it difficult for him to contribute to his own care and to maintain intimate personal relationships, even with members of his own family.

Under supervision of his physician, the patient receives secretin at a dosage of 8 μg/kg/day (that is, 560 μg/day) using subcutaneous administration.

Upon improvement in learning and memory in the Alzheimer's patient, the physician should regularly assess the patient, and administer secretin regularly to avoid recurrence of the patient's impaired learning memory symptoms.

### Example 9

### Reduction of Startle in a Bull

A 4 year old, 1500 kg, breeding bull is constantly injuring itself on the walls, door, and floor of its enclosure because it startles easily at loud noises and fast movements. Even sounds and movements made by the cows it is supposed to breed with easily startle the bull.

A veterinarian begins administering secretin to the bull at a dosage of 0.5 μg/kg/day (that is, 750 μg/day), injecting into the bull's shoulder.

Upon improvement in startle response in the bull, the veterinarian should regularly assess the bull, and administer secretin regularly to avoid recurrence of the bull's exaggerated startle response.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating an affective disorder in a patient, the method comprising administering to the patient an effective dosage of secretin or a secretin analog.

2. The method of claim 1, wherein the dosage of secretin or a secretin analog is effective to reduce one or more symptoms of anxiety, fear, impaired learning, impaired memory, apathy, delusions, anxiety, and autonomic changes, avoidance, increased arousal, depression, elevated mood, irritable mood, hallucinations, disorganized speech, and grossly disorganized behavior.

3. The method of claim 1, wherein the affective disorder is generalized anxiety disorder (GAD), social anxiety disorder (SAD; alternatively known as social phobia), panic disorder (with or without agoraphobia), posttraumatic stress disorder (PTSD), obsessive-compulsive disorder (OCD), separation anxiety disorder, a mood disorder, a psychotic disorder, a substance-related disorder, a cognitive disorder, an affective disorder associated with neurological medical disorders, or an affective disorder of childhood.

4. The method of claim 3, wherein the mood disorder is depressive disorder or bipolar disorder.

5. The method of claim 3, wherein the psychotic disorder is schizophrenia, schizoaffective disorder, or delusional disorder.

6. The method of claim 3, wherein the substance-related disorder is substance abuse, substance-induced disorder, or substance withdrawal.

7. The method of claim 3, wherein the cognitive disorder is dementia, delirium, or Alzheimer's type dementia.

8. The method of claim 3, wherein the affective disorder associated with neurological medical disorders is a seizure disorder or epilepsy.

9. The method of claim 3, wherein the affective disorder of childhood is attention disorder, attention deficit hyperactivity disorder, learning disorder, or separation anxiety.

10. The method of claim 3, wherein the substance is alcohol, an amphetamine, cocaine, nicotine, or an opioid.

11. The method of claim 1, wherein the secretin or secretin analog is administered subcutaneously, orally, nasally, parenterally, or intravenously.

12. The method of claim 1, wherein the secretin or secretin analog is administered multiple times.

13. The method of claim 12, wherein the times occur at regular intervals.

14. The method of claim 12, wherein the times occur intermittently.

15. The method of claim 1, wherein the patient suffers no adverse side-effects.

16. The method of claim 1, wherein the patient is a human, dog, cat, horse, donkey, cow, sheep, goat, pig, rat, or mouse.

17. The method of claim 1, wherein the effective dosage is 0.04 μg/kg, 0.5 μg/kg, 1 μg/kg, 3 μg/kg, 10 μg/kg, 30 μg/kg, 100 μg/kg, or 400 μg/kg.

18. The method of claim 1, wherein the secretin is human, bovine, equine, porcine, or canine secretin.

19. A method for reducing anxiety in a patient, the method comprising administering to the patient a dosage of secretin or a secretin analog effective to reduce anxiety in the patient.

20. The method of claim 19, wherein the patient has generalized anxiety disorder (GAD), social anxiety disorder (SAD; alternatively known as social phobia), panic disorder (with or without agoraphobia), posttraumatic stress disorder (PTSD), obsessive-compulsive disorder (OCD), separation anxiety disorder, a mood disorder, a psychotic disorder, a substance-related disorder, a cognitive disorder, an affective disorder associated with neurological medical disorders, or an affective disorder of childhood.

21. The method of claim 19, wherein the secretin or secretin analog is administered subcutaneously, orally, nasally, parenterally, or intravenously.

22. The method of claim 19, wherein the effective dosage is 0.04 μg/kg, 0.5 μg/kg, 1 μg/kg, 3 μg/kg, 10 μg/kg, 30 μg/kg, 100 μg/kg, or 400 μg/kg.

23. The method of claim 19, wherein the secretin is human, bovine, equine, porcine, or canine secretin.

24. A method for treating a patient having a disorder with an attendant symptom of anxiety, impaired learning, or impaired memory, the method comprising administering to the patient a dosage of secretin or a secretin analog effective to reduce an attendant symptom of anxiety, impaired learning, or impaired memory in the patient.

25. The method of claim 24, wherein the patient has generalized anxiety disorder (GAD), social anxiety disorder (SAD; alternatively known as social phobia), panic disorder (with or without agoraphobia), posttraumatic stress disorder (PTSD), obsessive-compulsive disorder (OCD), separation anxiety disorder, a mood disorder, a psychotic disorder, a substance-related disorder, a cognitive disorder, an affective disorder associated with neurological medical disorders, or an affective disorder of childhood.

26. The method of claim 24, wherein the secretin or secretin analog is administered subcutaneously, orally, nasally, parenterally, or intravenously.

27. The method of claim 24, wherein the effective dosage is 0.04 μg, 0.5 μg/kg, 1 μg/kg, 3 μg/kg, 10 μg/kg, 30 μg/kg, 100 μg/kg, or 400 μg/kg.

28. The method of claim 24, wherein the secretin is human, bovine, equine, porcine, or canine secretin.

29. A method for enhancing cognition in a patient, the method comprising administering to the patient a dosage of secretin or a secretin analog effective to enhance learning or memory or both in the patient.

30. The method of claim 29, wherein the patient suffers from impaired learning or memory.

31. The method of claim 29, wherein enhancement of learning is measured by improvement on a Wisconsin Card Sort Test.

32. The method of claim 29, wherein the secretin or secretin analog is administered subcutaneously, orally, nasally, parenterally, or intravenously.

33. The method of claim 29, wherein the effective dosage is 0.04 μg/kg, 0.5 μg/kg, 1 μg/kg, 3 μg/kg, 10 μg/kg, 30 μg/kg, 100 μg/kg, or 400 μg/kg.

34. The method of claim 29, wherein the secretin is human, bovine, equine, porcine, or canine secretin.

35. A kit comprising a composition comprising secretin or a secretin analog and instructions for use according to the method of claim 1.

36. A kit comprising a composition comprising secretin or a secretin analog and instructions for use according to the method of claim 19.

37. A kit comprising a composition comprising secretin or a secretin analog and instructions for use according to the method of claim 24.

38. A kit comprising a composition comprising secretin or a secretin analog and instructions for use according to the method of claim 29.
